(19) United States
(12) Patent Application Publication
(10) Pub. No.: US 2008/0032349 A1
(43) Pub. Date: Feb. 7, 2008

(54) METHOD FOR PRODUCING K5 POLYSACCHARIDE

(75) Inventors: Christian Viskov, Combs la ville (FR); Fabien Lux, Saint-Priest (FR); Regis Gervier, Lyon (FR); Gilles Colas, Alfortville (FR)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: Aventis Pharma S.A., Antony (FR)

(21) Appl. No.: 11/683,659

(22) Filed: Mar. 8, 2007

Related U.S. Application Data
(63) Continuation of application No. PCT/FR05/02210, filed on Sep. 6, 2005.

(30) Foreign Application Priority Data
Sep. 8, 2004 (FR) 0409497

(51) Int. Cl. C12P 19/04

(52) U.S. Cl. 435/101

(57) ABSTRACT

The invention concerns a method for producing K5 polysaccharide, comprising a fermentation step from Escherichia coli followed by a purification step.

The present invention relates to a process for producing polysaccharide K5, by performing a fermentation step with Escherichia coli followed by a purification step.

Processes for obtaining polysaccharide K5 are known. It may be obtained from E. coli strains responsible for extra-intestinal infections.

Polysaccharide K5 is composed of equimolar amounts of glucuronic acid and of N-acetylglucosamine, which constitute the 4-beta-glucuronyl-1,4-alpha-N-acetylglucosamine base unit linearly repeated in the chain of polysaccharide K5.

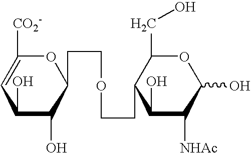

This base unit is structurally similar to completely desulfated and N-acetylated heparin, which makes it possible to envision a very advantageous alternative toward obtaining heparin by extraction of animal organs, i.e. the production of heparin by chemical modification of the polysaccharide K5 produced by fermentation.

Heparin obtained by biosynthesis is prepared in the following manner: the microorganism E. coli is cultured to obtain polysaccharide K5, which is then treated chemically to obtain heparin. Methods for preparing heparin from polysaccharide K5 are described (Biochem J., 1991, 275 (1):151-8; WO 92/17507; Semin. Thromb. Hemost., 2001, 27 (5) : 437-43; Nat. Biotech., 2003, 21 (11) : 1343-46). In general, for a synthesis of heparin from polysaccharide K5, deacetylation and N-resulfatation, C-5 epimerization and O-sulfatation must be envisioned.

Heparin is used especially for its anticoagulant and antithrombotic properties. However, heparin has drawbacks that limit the conditions of its use. In particular, its high anticoagulant activity (aIIa) may cause hemorrhaging (Semin. Thromb. Hemost., 5 sup. 3, 1999). According to the invention, by means of enzymatic or chemical depolymerization, it is possible, for example, to obtain from semisynthetic heparin low molecular weight heparins (3000-6500 Da) or very low molecular weight heparins (1500-3000 Da), for which the AntiXa activities are especially between 140 and 190 IU/mg for anti IIa activities of less than 5 IU/mg.

Numerous methods for preparing polysaccharide K5 are described in the prior art (Eur. J. Biochem, 1981, 116 : 359-364; EP0489 647, WO01/02597, Eur. J. Biochem, 1988, 117 : 112-125). They especially comprise an E. coli fermentation step consisting of a single growth phase in glucose at 37° C. followed by a step of purification of the polysaccharide K5, consisting essentially of treatment on an ion-exchange column.

The fermentation step of the process according to the present invention, for its part, comprises several growth phases, including at least one with feed, and a cooling phase, which make it possible to obtain, in combination with the purification step, yields up to 10 times greater than those described in the prior art.

Specifically, the polysaccharide K5 biosynthesis methods described in the prior art by fermentation of E. coli all have the drawback of having a very low yield. For example, the production of polysaccharide K5 in extracellular form according to the prior art produces an isolated polysaccharide K5 in yields of only from 200 to 850 mg/l.

Such low yields do not make it possible to envision an industrial production of polysaccharide K5 and then, via semisynthesis, of semisynthetic heparin. Now, there is a great need to obtain heparin and also low molecular weight or very low molecular weight heparins of non-animal origin.

One subject of the invention is thus a novel method for preparing polysaccharide K5 in extracellular form.

The process according to the invention comprises essentially two steps: a fermentation step and a purification step.

According to the process of the invention, it is possible to obtain polysaccharide K5 concentrations up to 10 times greater than those obtained with the methods described in the prior art. It is thus possible according to the invention to obtain up to 10 g/l of polysaccharide K5. The production of such concentrations is associated with the combination of the fermentation and purification steps.

### BRIEF DESCRIPTION OF THE DRAWINGS

FIG. 1 illustrates the Amount of exploitable polysaccharide for the COL5K000 to COL5K006 operations. The solid bars represent the estimated exploitable amount of polysaccharide (in g); the dotted bars represent the final concentration of polysaccharide in the supernatant (×20, in g/l); the triangles represent the OD-600 nm achieved by the cultures.

FIG. 2 illustrates the profile of the main parameters of the COL5K006 operation. Curve 1 represents the percentage of dissolved oxygen in the medium, curve 2 represents the rate of production of CO₂ in the culture COLK006 (in nmol.l⁻¹h⁻¹), curve 3 represents the volume fed/20 in g.l⁻¹, and curve 4 represents the concentration of polysaccharide K5 in the supernatant (in g.l⁻¹).

One subject of the invention is a process for preparing polysaccharide K5, comprising:
a step of fermentation, which may be performed in a fermenter, of a strain of Escherichia coli that produces polysaccharide K5, comprising:
   a batch growth phase,
   one or more fed batch growth phases,
   a cooling phase, during which the temperature is decreased and the pH of the medium rises to a value that may be up to 9,
   a phase of separating the biomass from the culture medium,
   a phase of decontaminating the culture medium from the biomass,
and a purification step using the culture medium and/or the biomass.
A subject of the invention is, more particularly, the process for preparing polysaccharide K5 as described above, the purification step of which starting with the culture medium and/or the biomass does not comprise a step of treatment on an ion-exchange column, but comprises, in the following order:
   adjustment of the pH to between about 7 and about 11,
   precipitation,
   dissolution of the precipitate,
   filtration of the resulting suspension, and
   precipitation from an alcohol.
      Fermentation

The E. coli strains that may be used for the production of polysaccharide K5 according to the invention may be obtained from public collections, such as the CNCM (Collection Nationale de Cultures de Microorganismes, France) or the ATCC (American Type Culture Collection, USA). Alternatively, these strains may be obtained via clinical isolation followed by characterization of the antigenic type of the capsule, as described in the literature (Eur. J. Biochem, 1988 117 : 112-125).

The fermentation step according to the invention is performed using the microorganism E. coli in a fermenter, and comprises:
a batch growth phase,
one or more fed batch growth cycles, which may be with exponential or constant feed,
a cooling phase, during which the temperature may be decreased to about 10-12° C. and the pH rises to about 7.5-9,
a phase of separating the biomass from the culture medium, and
a phase of decontamination of the culture medium from the biomass.

According to the invention, the expression “batch growth” means a growth of a microorganism in a reactor without feed or removal.

According to the invention, the expression “fed batch growth” means a growth of a microorganism in a reactor with controlled feed of substrate and without removal.

The fermentation step according to the invention in particular comprises two batch growth phases with exponential feed of substrate, which follow the batch growth phase.

The fermentation step according to the invention comprises a batch growth phase with constant feed of substrate, preceding the cooling phase.

In the fermentation step according to the invention, the first fed batch growth phase begins on depletion of the carbon source in the medium during the batch growth phase.

More particularly, according to the invention, the first fed batch growth phase begins on depletion of the carbon source in the medium during the batch growth phase and, where appropriate, the following fed batch growth phases begin when the concentration of dissolved oxygen drops significantly as a result of a limitation of the oxygen transfer.

According to the invention, each of the batch growth phases begins as described above and the cooling phase begins when all the culture feed medium has been injected into the fermenter, or when a change in pH is detected.

According to the invention, after the batch growth phase, the fed batch growth phase(s) and the cooling phase, the biomass is separated from the medium by centrifugation. After separation, the biomass and the culture medium are decontaminated; in particular, they are inactivated over a period ranging between 1 hour and 3 hours at a temperature of about 80° C. The supernatant and/or the pellet is (are) then used for the purification of polysaccharide K5.

The pH is maintained at about 6.5 during the batch growth phase and the fed batch growth phase, by addition of NH₄OH or NH₃, and is raised to about 8.5 during the final phase.

The dissolved oxygen concentration is maintained between about 15% and about 80% of air saturation by varying the stirring speed, the air flow rate, the oxygen enrichment of the inlet air or by adding hydrogen peroxide.

The growth temperature during the fermentation step according to the invention is between about 10° C. and about 40° C. It is more particularly about 30° C. during the batch growth phase and the first fed batch growth phase, and about 25° C., where appropriate, during the other fed batch growth phases. Passing from 30° C. to 25° C. is particularly important for obtaining high yields of polysaccharide K5.

The culture medium used is a synthetic culture medium. The carbon source according to the invention is either glucose or glycerol. The preferred carbon source according to the invention is glycerol.

More particularly, the carbon source in the fermentation step according to the invention is glycerol at a concentration of between about 10 and about 90 g/l in the initial culture medium for the batch growth phase, and between about 250 and about 1200 g/l in the feed medium for the fed batch growth phases. Experimental example 1 illustrates the importance of the use of glycerol as carbon source and of increasing the glycerol concentration in the feed medium to obtain high concentrations of polysaccharide K5.

The nitrogen source in the medium during the batch growth phase according to the invention may be yeast extracts, casamino acids, peptones, NH₄OH, NH₃ or (NH₄)₂HPO₄.

According to the invention, the nitrogen source in the initial culture medium during the batch growth phase is more particularly NH₄OH, NH₃ or (NH₄)₂HPO₄. It is used in particular at a concentration of between about 1 and about 10 g/l.

During the batch growth and fed batch growth phases according to the invention, the nitrogen source is provided by NH₄OH or NH₃ for regulation of the pH. Its concentration is preferably about 20%.

More particularly, in the fermentation step according to the invention, the nitrogen source in the medium during the batch growth phase is NH₄OH, NH₃ or (NH₄)₂HPO₄, at a concentration of between about 1 and about 10 g/l in the initial culture medium, and is provided by NH₄OH or NH₃ at a concentration of about 20% for regulation of the pH during the batch growth and fed batch growth phases.

A subject of the invention is the process for preparing polysaccharide K5 as described above, comprising a fermentation step in which:
the carbon source is glycerol at a concentration of between:
   about 10 and about 90 g/l in the initial culture medium for the batch growth phase, and
   about 250 and about 1200 g/l in the feed medium for the fed batch growth phases, and
the growth temperature is:
   about 30° C. during the batch growth phase and the first fed batch growth phase, and
   about 25° C., where appropriate, during the other fed batch growth phases.

A subject of the invention is also the process for preparing polysaccharide K5 as described above, comprising a fermentation step in which:
the carbon source is glycerol at a concentration of between:
   about 10 and about 90 g/l in the initial culture medium for the batch growth phase, and
   about 250 and about 1200 g/l in the feed medium for the fed batch growth phases; and
the nitrogen source in the medium during the batch growth phase:
   is NH₄OH, NH₃ or (NH₄)₂HPO₄, at a concentration of between about 1 and about 10 g/l in the initial culture medium, and
   is provided by NH₄OH or NH₃ at a concentration of about 20% for regulation of the pH during the batch growth and fed batch growth phases.

A subject of the invention is also the process for preparing polysaccharide K5 as described above, comprising a fermentation step in which:
the nitrogen source in the medium during the batch growth phase:
   is NH₄OH, NH₃ or (NH₄)₂HPO₄, at a concentration of between about 1 and about 10 g/l in the initial culture medium, and
   is provided by NH₄OH or NH₃ at a concentration of about 20% for regulation of the pH during the batch growth and fed batch growth phases; and
the growth temperature is:
   about 30° C. during the batch growth phase and the first fed batch growth phase, and
   about 25° C., where appropriate, during the other fed batch growth phases.

A subject of the invention is, most particularly, the process for preparing polysaccharide K5 as described above, in which:
the fermentation step comprises the following steps, in the following order:
   a batch growth phase,
   a batch growth phase with exponential feed of substrate starting from the total depletion of the carbon source during the preceding phase,
   a second batch growth phase with exponential feed of substrate starting when the oxygen concentration drops significantly as a result of a limitation of the oxygen transfer in the culture medium during the preceding phase,
   a batch growth cycle with constant feed of substrate starting when the oxygen concentration drops significantly as a result of a limitation of the oxygen transfer in the culture medium during the preceding phase, and
   a cooling phase during which the temperature is decreased to about 12° C. and the pH rises to a value of between about 7.5 and about 9,
   a phase of decontamination of the culture medium from the biomass,
and is performed with the following parameters:
   the temperature is:
      about 30° C. during the batch growth and the first fed batch growth phase, and
      about 25° C. during the other two fed batch growth phases;
   the nitrogen source is provided by:
      (NH₄)₂HPO₄, NH₄OH or NH₃ at a concentration of between about 1 and about 10 g/l in the initial culture medium,
      the addition of NH₄OH or concentrated NH₃ to about 20% for regulation of the pH during the batch growth and fed batch growth phases,
   the carbon source is glycerol:
      at a concentration of between about 10 and about 90 g/l in the initial culture medium during the first batch growth phase, and
      at a concentration of between about 250 and about 1200 g/l in the feed medium during the fed batch growth phases.
         Second Step: Purification of Polysaccharide K5

A subject of the invention is also the process for preparing polysaccharide K5 as described above, comprising a purification step during which the pH of the solution containing the polysaccharide K5, and which may be the centrifugation supernatant from the fermentation broth or the resuspended pellet from this centrifugation, is adjusted to between about 7 and about 11, and the polysaccharide is precipitated from this solution. The precipitate is then washed, redissolved and filtered, and then precipitated.

According to the invention, the first precipitation of the purification step takes place using a quaternary ammonium salt. This precipitation takes place more particularly using benzethonium chloride.

In the purification step according to the invention, the first precipitate is redissolved in sodium acetate, used especially at a concentration of 10%.

Moreover, methanol is used to precipitate the filtrate in the purification step according to the invention.

More particularly, the first precipitation of the purification step according to the invention is performed using benzethonium chloride, the precipitate is dissolved in sodium acetate and then filtered, and the filtrate from the purification step is precipitated from methanol.

According to the invention, the purified product as indicated above may optionally undergo one or more decolorizations with hydrogen peroxide. It in particular undergoes three treatments with hydrogen peroxide after the purification.

Where appropriate, the purified product as indicated above undergoes, after the three decolorizations with hydrogen peroxide, an additional treatment with hydrogen peroxide to remove the remaining contaminations. This treatment allows an improvement in the purity of the product obtained. A subject of the invention is thus also a process for preparing polysaccharide K5, comprising another decolorization with hydrogen peroxide after purification and three treatments with hydrogen peroxide.

Where appropriate, the polysaccharide K5 is treated with a protease after any one of the above-mentioned purification steps. As indicated later in experimental example 8, the treatment with protease makes it possible to significantly increase the purity of the polysaccharide K5 produced in an almost quantitative yield. A subject of the invention is thus, in particular, a process for preparing polysaccharide K5, comprising a treatment with a protease during the purification step.

The protease according to the invention is preferably alcalase.

Finally, the polysaccharide K5 is precipitated, centrifuged and dried.

A subject of the invention is thus, most particularly, a process for preparing polysaccharide K5 as described above, comprising a purification step during which the following steps are performed:
centrifugation of the culture to separate the culture medium from the biomass;
using the supernatant from the centrifugation of the fermentation broth for:
   adjustment of the pH to between about 7 and about 11,
   precipitating with benzethonium chloride,
   washing the precipitate,
   dissolving the precipitate in 10% sodium acetate,
   filtering the 10% sodium acetate solution,
   precipitating the filtrate from methanol,
   decolorizing the filtrate three times with hydrogen peroxide,
   treating with alcalase;
in parallel, using the pellet resulting from the centrifugation of the fermentation broth for:
   resuspending the pellet,
   adjusting the pH to between about 7 and about 11,
   precipitating with benzethonium chloride,
   washing the precipitate,
   redissolving the precipitate in 10% sodium acetate,
   filtering the 10% sodium acetate solution,
   precipitating the filtrate from methanol,
   decolorizing the filtrate three times with hydrogen peroxide,
   treating with alcalase;
combining the two preparations in any one of the purification steps;
precipitating, centrifuging and drying the polysaccharide K5.

A subject of the invention is thus, preferably, a process for preparing polysaccharide K5, comprising:
a fermentation step:
   which comprises the following steps, in the following order:
      a batch growth phase,
      a batch growth phase with exponential feed of substrate starting from the total depletion of the carbon source during the preceding phase,
      a second batch growth phase with exponential feed of substrate starting when the oxygen concentration drops significantly as a result of a limitation of the oxygen transfer in the culture medium during the preceding phase,
      a batch growth cycle with constant feed of substrate starting when the oxygen concentration drops significantly as a result of a limitation of the oxygen transfer in the culture medium during the preceding phase, and
      a cooling phase during which the temperature is decreased to about 12° C. and the pH rises to a value of between about 7.5 and about 9,
      a phase of decontamination of the culture medium from the biomass,
   and which is performed with the following parameters:
      the temperature is:
         about 30° C. during the batch growth and the first fed batch growth phase, and
         about 25° C. during the other two fed batch growth phases;
      the nitrogen source is provided by:
         (NH₄)₂HPO₄, NH₄OH or NH₃ at a concentration of between about 1 and about 10 g/l in the initial culture medium,
         the addition of NH₄OH or concentrated NH₃ to about 20% for regulation of the pH during the batch growth and fed batch growth phases,
      the carbon source is glycerol:
         at a concentration of between about 10 and about 90 g/l in the initial culture medium during the first batch growth phase, and
         at a concentration of between about 250 and about 1200 g/l in the feed medium during the fed batch growth phases;
a purification step during which the following steps are performed:
   centrifugation of the culture to separate the culture medium from the biomass;
   using the supernatant from the centrifugation of the fermentation broth for:
   adjustment of the pH to between about 7 and about 11,
   precipitating with benzethonium chloride,
   washing the precipitate,
   dissolving the precipitate in 10% sodium acetate,
   filtering the 10% sodium acetate solution,
   precipitating the filtrate from methanol,
   decolorizing the filtrate three times with hydrogen peroxide,
   treating with alcalase;
   in parallel, using the pellet resulting from the centrifugation of the fermentation broth for:
   resuspending the pellet,
   adjusting the pH to between about 7 and about 11,
   precipitating with benzethonium chloride,
   washing the precipitate,
   redissolving the precipitate in 10% sodium acetate,
   filtering the 10% sodium acetate solution,
   precipitating the filtrate from methanol,
   decolorizing the filtrate three times with hydrogen peroxide,
   treating with alcalase;
   combining the two preparations in any one of the purification steps;
   precipitating, centrifuging and drying the polysaccharide K5.

The process according to the invention thus makes it possible to obtain yields that may be up to 10 times greater than the yields obtained with the methods described in the prior art.

According to the invention, the polysaccharide K5 produced as described above is then used as substrate for a chemical and/or enzymatic deacetylation, N-resulfatation, C-5 epimerization and/or O-sulfatation reaction. It is more particularly used according to the invention as substrate in one of the abovementioned reactions to obtain a semisynthetic heparin (bioheparin).

The bioheparin obtained is then used according to the invention as substrate in a fragmentation reaction to obtain an LMWH (low molecular weight heparin), the molecular weight of which is between about 1500 and about 6500 Da.

It is also possible according to the invention to use the bioheparin obtained as substrate in a fragmentation reaction to obtain an LMWH (low molecular weight heparin), the molecular weight of which is more particularly between about 3500 and about 5500 Da, or a VLMWH (very low molecular weight heparin), the molecular weight of which is more particularly between about 1500 and about 3000 Da.

The examples that follow illustrate the invention without, however, limiting it.

### APPLICATION EXAMPLES

### Example 1

1) Procedure

a) Fermentation

The fermentation is performed using the E. coli strain ATCC23506 in a 20-liter fermenter.

A preculture of the E. coli strain is prepared using 1.8 ml of a vial frozen overnight at 30° C. in a 500 ml conical flask containing 100 ml of the following medium: glycerol 5 to 60 g.l⁻¹, KH₂PO₄ 0.5 to 3 g.l⁻¹, (NH₄)₂HPO₄ 1 to 10 g.l⁻¹, MgSO₄ 0.5 to 5 g.l⁻¹, citric acid 0.2 to 4 g.l⁻¹, and also trace elements.

The growth takes place at 30° C. with stirring at 200 rpm. This preculture is used to inoculate the production fermenter. The fermenter may also be inoculated directly from the glycerol-containing vial, the object of the preculture being only to reduce the duration of the batch growth phase in the fermenter. The media used are variants of the RFB MIL 11/04 medium (J. of Biotechnology, 1995, 39 : 59-65) and their compositions are given in table I.

TABLE I
Composition of the media used in batch growth and
in batch growth with controlled feed of substrate.

| | | Medium for batch |
|---|---|---|
| | Medium for batch | growth with feed of |
| | growth, per liter | substrate, per liter |
| Materials | of medium | of medium |
| Glycerol | Between 10 and 90 g | Between 250 and 1000 |
| or glucose | (preferably 30 g) | (preferably 750 g) |
| | | for glycerol |
| | | Between 250 and 700 g |
| | | (preferably 500 g) |
| | | for glucose |
| Citric acid | Between 0.5 and 3 g | — |
| | (preferably 1.7 g) | |
| KH₂PO₄ | Between 5 and 20 g | Between 5 and 37.5 g |
| | (preferably 13.3 g) | (preferably 15 g) |
| (NH₄)₂HPO₄ | Between 1 and 10 g | — |
| | (preferably 4 g) | |
| Fe(III) citrate | Between 20 and | Between 10 and |
| | 200 mg (preferably | 62.5 mg (preferably |
| | 87.5 mg) | 30 mg) |
| EDTA | Between 2 and 20 mg | Between 2.5 and 25 mg |
| | (preferably 8.4 mg) | (preferably |
| | | 10.075 mg) |
| CoCl₂•6 H₂O | Between 1 and 15 mg | Between 1.25 and |
| | (preferably 2.5 mg) | 17.5 mg (preferably |
| | | 3.0 mg) |
| MnCl₂•4 H₂O | Between 5 and 50 mg | Between 5 and 25 mg |
| | (preferably 15 mg) | (preferably 18.0 mg) |
| CuCl₂•2 H₂O | Between 1 and 10 mg | Between 1.25 and |
| | (preferably 1.5 mg) | 12.5 mg (preferably |
| | | 1.8 mg) |
| H₃BO₃ | Between 1 and 15 mg | Between 1.25 and |
| | (preferably 3 mg) | 17.5 mg (preferably |
| | | 3.6 mg) |
| Na₂Mo₄•2 H₂O | Between 1 and 15 mg | Between 1.25 and |
| | (preferably 2.5 mg) | 17.5 mg (preferably |
| | | 3.0 mg) |
| Zn(CH₃COO)₂•2 H₂O | Between 5 and 30 mg | Between 5 and 37.5 mg |
| | (preferably 13 mg) | (preferably 15.6 mg) |
| Fe(III) citrate | Between 5 and 30 mg | Between 5 and 37.5 mg |
| | (preferably 12.5 mg | (preferably 15.0 mg) |
| Demineralized H₂O | qs. 1000 ml | qs. 1000 ml |
| MgSO₄•7 H₂O | Between 0.5 and | Between 6.25 and 50 g |
| | 10 g (preferably | (preferably 15 g), |
| | 1.2 g) | |
| Thiamine HCl | Between 5 and 50 mg | — |
| (vit B1) | (preferably 15 mg) | |

The cultures are prepared in TECHFORS fermenters (20 l total volume, 12 l working volume) controlled by the IRIS program (Infors) based on a self-adaptative control algorithm. The dissolved oxygen concentration in the medium, the pH, the stirring speed, the temperature, the flow rate of aerating air, the pressure and the pump deliveries (basic regulation, feed, antifoam) are measured and controlled online. The analysis of the fermenter outlet gases is performed using a mass spectrometer (PRIMA 600S).

The fermentation is performed in batch mode with the following characteristics:
working volume of 12 l,
initial volume of 8 l,
volume added of 4 l,
final volume of 12 l plus the base volume,
dissolved oxygen maintained between 15% and 80% of air saturation (preferably maintained at 30%),
temperature between 20 and 40° C. (preferably 30° C.),
pH regulated to between 6.0 and 7.5 (preferably maintained at 6.5) by addition of concentrated NH₄OH or NH₃,
pressure of 0.3 bar, 0.5 bar and 0.8 bar,
initial aeration of 4 l.min⁻¹ (0.5 VVM) (regulated to between 4 and 20 l.min⁻¹), and
initial stirring speed of 200 rpm (regulated to between 200 and 1500 rpm).

The culturing may comprise up to five steps: a batch growth phase followed by three different batch growth steps with feed of substrate, and a final cooling step.

1. Batch Growth Phase

During the culturing phase in batch mode, the strain generation time is estimated from measurement of the production of carbon dioxide and the initial rate of the first part of the batch growth with controlled feed of substrate is calculated:
Q₀=(CO₂ₚₘₐₓ×Vol_{Fer})/(1000×MW_{CO2}×Y_{CO2/glycerol}×G₀)
Q₀: initial feed rate of concentrated medium (l.h⁻¹); CO₂ₚₘₐₓ: maximum production of carbon dioxide (mmol.l⁻¹.h⁻¹); G₀: concentration of glycerol or glucose in the concentrated medium used during the batch growth with controlled feed of substrate (750 g.l⁻¹); Vol_{Fer}: volume of liquid in the fermenter; MW_{CO2}: molecular weight of CO₂ (g.l⁻¹) and Y_{CO2/glycerol}: yield of CO₂ produced per amount of glycerol consumed.

2. First Batch Growth Phase with Exponential Feed of Substrate

After depletion of the carbon source, a batch growth cycle with exponential feed of substrate is initiated by feeding with concentrated medium, in order to increase the concentration of biomass. The pump feeding fresh medium is switched on. Its delivery follows an exponential increase over time:
Q(t)=Q₀×2^{[t/(τ×2.4)]}
Q₀: initial feed rate (l.h⁻¹) of the feed pump; t: time elapsed from the start of the batch growth cycle with exponential feed of substrate; τ: strain generation time, measured during the batch growth.

This feed rate takes into account the generation time previously measured multiplied by a correction factor that may range from 1 to 10 (preferably 2.4). This feed rate imposes on the microorganism a specific growth rate of between 0.05 h⁻¹ and 0.65 h⁻¹.

3. Second Batch Growth Phase with Exponential Feed of Substrate

At the first limitation of oxygen transfer (dissolved oxygen<20%, stirring speed>1350 rpm, air flow rate>19.5 l.min⁻¹, pressure=0.5 bar), this first batch growth cycle with exponential feed of substrate is stopped. Another exponential feed is started on the basis of an initial rate of 8.4 ml.h⁻¹ at the start of the first batch growth cycle with controlled feed of substrate. The generation time is multiplied by a new correction factor of between 4 and 10 (preferably 6) as during the preceding batch growth cycle with exponential feed of substrate. This feed rate imposes on the microorganism a specific growth rate of between 0.05 h⁻¹ and 0.16 h⁻¹.

At the same moment, the nominal temperature of the fermenter is lowered to between 20 and 25° C. (preferably 25° C.) in order to increase the oxygen solubility in the medium. The new control algorithm of the feed pump is:
Q′(t)=0.0084×2^{[t/(τ×6)]}
t: time elapsed since the start of the batch growth cycle with exponential feed of substrate; τ: strain generation time measured during the batch growth.

Whereas this limitation of oxygen transfer appears at about 30 h of growth (including the 10 h of the first batch growth cycle with controlled feed of substrate), the real initial delivery of medium of the feed pump is about 15 ml.h⁻¹ (t=10 h). This lower feed rate once again allows the system to regulate the dissolved oxygen to 30% of saturation while at the same time maintaining the growth of the microorganism. This feed continues until a second limitation of oxygen transfer appears.

4. Phase of Constant Feed of Substrate

On appearance of the second limitation of oxygen transfer (dissolved oxygen<20%, stirring speed>1350 rpm, air flow rate>19.5 l.min⁻¹, pressure=0.5 bar), the exponential feed of medium is converted into a constant delivery of between 1 and 20 ml.l⁻¹.h⁻¹ (preferably 4.2 ml.l⁻¹.h⁻¹). This feed continues until the 4 l of feed medium have been injected or until a significant drop in pH is detected (total consumption of the NH₄OH, indicating a strong limitation of oxygen).

5. Cooling Phase

The feed pump is then switched off and the fermenter temperature is brought to 10° C., while the pH naturally rises or may be adjusted to a value of between 7.5 and 9 (preferably 8.5), which is favorable toward the activity of the enzyme polysaccharide K5 lyase. These conditions are maintained for a period of from 0 to 10 h.

After the fermenter has been cooled to 10° C., the biomass is harvested under level-2 biological hazard working conditions, in a hermetic container. The broth is centrifuged to separate the biomass from the culture supernatant. After separation, the supernatant is decontaminated with stirring by means of a heat treatment at 80° C. for 1 hour 30 min. The biomass is itself decontaminated by treatment at 80° C. for 2 hours 30 min. The supernatant and the biomass may be stored at −20° C.

The conditions used in these tests are:
COL5K000/001: batch growth phase at 30° C./batch growth phase with exponential feed of substrate at 30° C./cooling phase.
COL5K002: batch growth phase at 30° C./batch growth phase with exponential feed of substrate at 30° C./second batch growth phase with exponential feed of substrate at 30° C./cooling phase.
COL5K003/04: batch growth phase at 30° C./batch growth phase with exponential feed of substrate at 30° C./second batch growth phase with exponential feed of substrate at 25° C./cooling phase.
COL5K005/006: batch growth phase at 30° C./batch growth phase with exponential feed of substrate at 30° C./second batch growth phase with exponential feed of substrate at 25° C./batch growth phase with constant feed of substrate at 25° C./cooling phase.

b) Analysis of the Polysaccharides

The analysis of the polysaccharides is performed in two stages:
1. the polysaccharides are first depolymerized to their constituent disaccharides,
2. the disaccharide mixture is analyzed by HPLC.

1. Depolymerization of the Polysaccharides

About 40 mg of sample to be analyzed are resuspended in 2 ml of pH 7 acetate buffer (0.29 ml of acetic acid, 5 mg of BSA, 15.8 mg of calcium acetate, demineralized water, qs 50 ml, pH adjusted to 7 with 2N sodium hydroxide) in order to obtain a solution containing 20 mg/ml.

At the same time, heparinase I, II and III solutions containing 0.5 IU/ml in phosphate buffer (68 mg of potassium dihydrogen phosphate, 10 mg of BSA, demineralized water, qs 50 ml, pH adjusted to 7 with 1N potassium hydroxide) are prepared. These solutions are then mixed in equal proportions to obtain the final heparinase 123 solution.

25 μl of the sample solution at 20 mg/ml, 25 μl of acetate buffer and 25 μl of heparinase 123 solution are then mixed together in a 300 μl tube. The mixture is homogenized thoroughly and incubated for 24 hours at room temperature, so as to obtain total depolymerization.

2. Analysis of the Polysaccharides by HPLC

The preceding mixture is analyzed by gradient HPLC according to the method well known to those skilled in the art. The analysis conditions are:
column: Spherisorb SAX 5 μm, 250×3 mm, Waters
column temperature: 40° C.
flow rate: 0.5 ml/min
injection volume: 10 μl
UV detection at 234 nm
mobile phase:
   solvent A: NaH₂PO₄ 2.5 mM pH=2.9
   solvent B: NaClO₄ M, NaH₂PO₄ 2.5 mM pH=3

Gradient:

| Time (min) | % B |
|---|---|
| 0 | 3 |
| 20 | 35 |
| 50 | 100 |
| 60 | 100 |
| 61 | 3 |
| 70 | 3 |

The disaccharide 4-beta-glucuronyl-1,4-alpha-N-acetylglucosamine is detected by HPLC. 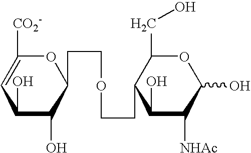
2) Results

Six different fermentation tests (COL5K001 to 006) were performed, in addition to the control, COL5K000.

The polysaccharide K5 concentrations obtained during these tests are collated in table 2.

TABLE 2

| | | | | | | | Total | | [K5] | Exploitable |
|---|---|---|---|---|---|---|---|---|---|---|
| | | Inoculums | | UO | Doubling | | duration | | supernatant | K5: |
| Test | Inoculation | OD | Medium | (I) | time (h) | Temperature | (h) | OD_{600 nm} | (g/l) | estimation |
| COL5 | 100 ml PC | 6.9 | RFB MIL 11/04 + | 8 + 4 | 1.1 | 30° C. | 45.1 | 218.5 | 1.0 | 24 |
| K000 | | | Glucose 30 g/l | | | | | | | |
| COL5 | 100 ml PC | 5.4 | RFB MIL 11/04 + | 8 + 4 | 1.5 | 30° C. | 45.8 | 176 | 3.8 | 54 |
| K001 | | | Glycerol 30 g/l | | | | | | | |
| COL5 | 100 ml PC | 6.9 | RFB MIL 11/04 + | 8 + 4 | 1.4 | 30° C. | 48.1 | 207.4 | 3.9 | 52 |
| K002 | | | Glycerol 30 g/l | | | | | | | |
| COL5 | 100 ml PC | 0.3 | RFB MIL 11/04 + | 8 + 4 | 1.4 | 30° C. + | 64.2 | 217 | 7.5 or | 85 |
| K003 | | | Glycerol 30 g/l | | | 25° C. | | | 6.9 | |
| COL5 | 100 ml PC | 1.5 | RFB MIL 11/04 + | 8 + 4 | 1.3 | 30° C. + | 68.4 | 193 | 2.1 | 23 |
| K004 | | | Glycerol 30 g/l | | | 25° C. | | | | |
| COL5 | 100 ml PC | 3.1 | RFB MIL 11/04 + | 8 + 4 | 1.4 | 30° C. + | 70.1 | 264 | 8.9 | 96 |
| K005 | | | Glycerol 30 g/l (fed- | | | 25° C. | | | | |
| | | | batch at 750 g/l gly) | | | | | | | |
| COL5 | 100 ml PC | 2.7 | RFB MIL 11/04 + | 8 + 4 | 1.5 | 30° C. + | 71 | n.a. | 10.2 | 103 |
| K006 | | | Glycerol 30 g/l (fed- | | | 25° C. | | | | |
| | | | batch at 750 g/l gly) | | | | | | | |

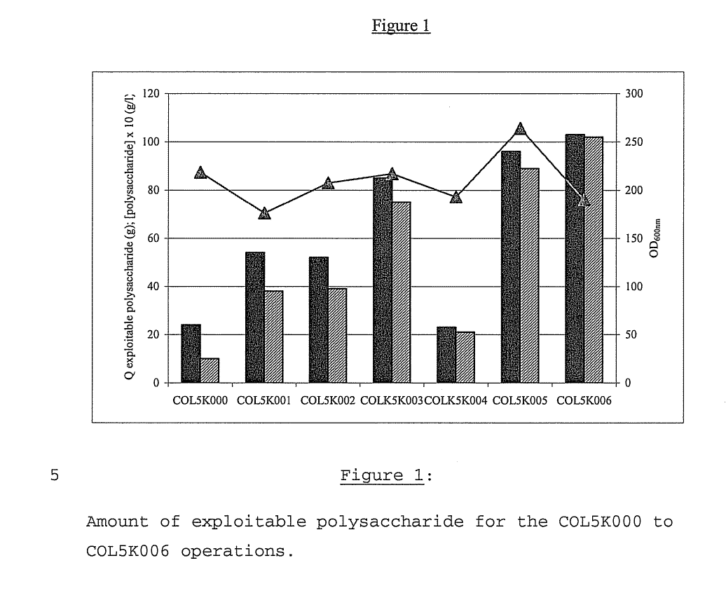

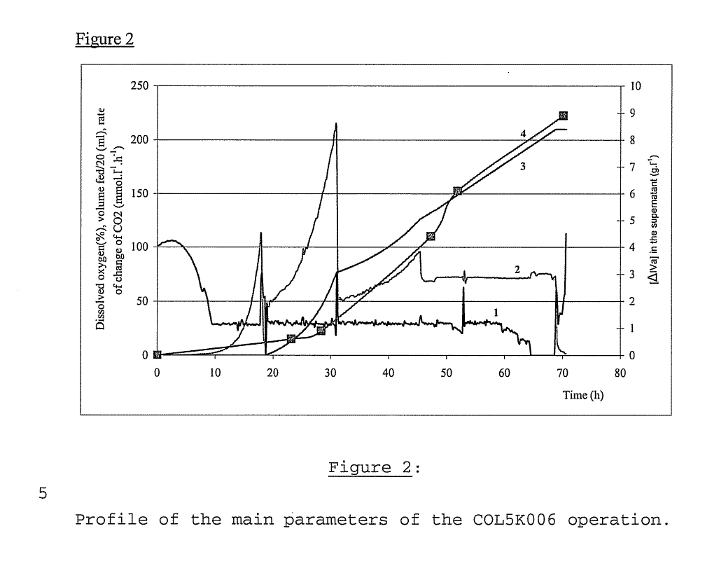

The results (FIG. 1, appendix 1) clearly show that each of the procedures used made it possible to produce between 2 and 10 times more polysaccharide K5 than the control COL5K000. The profile of the main parameters of COLK006 shows that the increase in the polysaccharide K5 concentration in the medium is associated with entry into the second fed batch growth phase (FIG. 2, appendix 1).

The study performed made it possible to greatly increase the maximum concentration of polysaccharide produced in the medium compared with that which has been described in the literature: the literature amount was 900 mg.l⁻¹, whereas we have obtained a concentration of greater than 10 g.l⁻¹.

The main changes made to the process between the operations COL5K000 and COL5K006 that made it possible to increase the polysaccharide concentration in the culture medium were:
the use of glycerol instead of glucose as carbon source,
increasing the total fermentation time, by adding sequences of feeding substrate at a reduced specific growth rate (second and third feed sequence) during the appearance of the limitation of oxygen transfer,
increasing the glycerol concentration of the feed solution (passing from 500 g.l⁻¹ to 750 g.l⁻¹), and
increasing the pH at the end of the operation, which makes it possible to establish conditions favorable toward the activity of polysaccharide K5 lyase and to increase the amount of polysaccharide available in the culture medium.

### Example 2

Extraction Process

The extraction steps described below were performed in order to isolate polysaccharide K5 from the fermentation supernatant obtained above.

6.5 liters of heat-inactivated supernatant were obtained in example 1, which contain 24.7 g of polysaccharide K5, from the fermenter COL5K001.

The pH of this solution was adjusted to 7.2 using 5N sodium hydroxide and 663 ml of an aqueous solution containing 50 g/l of benzethonium chloride were poured in (i.e. 1.2 molar equivalents of benzethonium chloride relative to the polysaccharide K5). The reaction medium was stirred at room temperature for 30 minutes and then centrifuged at 4000 rpm for 10 minutes.

The pellet obtained was washed twice with 4 liters of demineralized water, and was then stirred in 5 liters of 10% sodium acetate solution.

The supernatant obtained in the first precipitation with benzethonium chloride was reprocessed: its pH was adjusted to 7.2 using 5N sodium hydroxide, 663 ml of benzethonium chloride at a concentration of 50 g/l were added, and the mixture was stirred for 30 minutes before being centrifuged.

The pellet obtained was washed twice with 4 liters of demineralized water and was then stirred in 5 liters of 10% sodium acetate solution. The solutions containing the two resuspended pellets were combined and then filtered through a sinter funnel equipped with a Clarcel prelayer.

The filtrate was poured into 40 liters of methanol. The resulting mixture was stirred for 25 minutes, after which it was decanted overnight. The next day, it was centrifuged at 4000×g for 10 minutes. The pellet was washed with methanol and then dried for 36 hours at 50° C. under 20 Torr.

31.5 g of polysaccharide K5 with a titer of 54% were thus obtained; the yield was 68.8%.

### Example 3

Purification

Since the titer of the product isolated in example 2 was low (only 54%), additional purification steps were performed.

The crude product obtained in example 2, which weighs 31.5 g, was resuspended with 1.2 liters of demineralized water in a conical flask with magnetic stirring. After adjusting the pH to 11 with 5N sodium hydroxide, 15 g of sodium chloride and 6 ml of 30% hydrogen peroxide were added to the solution. The mixture was stirred for 10 minutes and then left overnight at room temperature without stirring.

The polysaccharide K5 was precipitated by adding 4 volumes of methanol to the solution; the mixture was stirred for 30 minutes and then centrifuged at 4000 rpm for 15 minutes. The pellet was oven-dried for 36 hours at 45° C. under 20 Torr. 23.5 g of polysaccharide K5 were thus obtained, with a titer that was improved from 54% to 65%.

### Example 4

Extraction and Purification

The crude polysaccharide K5 was extracted and purified from the fermenter COL5K002 of example 1.

5.4 liters of heat-inactivated supernatant were obtained in example 1, which contain 21.1 g of polysaccharide K5, from the fermenter COL5K002.

The COL5K002 supernatant was treated with benzethonium chloride and the pellets were stirred in an acetate solution and then filtered through a sinter funnel equipped with a Clarcel prelayer, as described in example 2.

The polysaccharide K5 contained in the filtrate was precipitated from methanol, as described in example 2, and was then subjected to decolorization with hydrogen peroxide, as detailed in example 3, followed by a second decolorization with hydrogen peroxide.

25.3 g of polysaccharide K5 were finally obtained, with a titer of 78% and a yield of greater than 90%.

### Example 5

Extraction and Purification

The crude polysaccharide K5 was extracted and purified from the fermenter COL5K003 of example 4.

6 liters of heat-inactivated supernatant containing 45 g of polysaccharide K5 were obtained in example 1, from the fermenter COL5K003.

The COL5K003 supernatant was treated with benzethonium chloride and the pellets were stirred in an acetate solution and then filtered through a sinter funnel equipped with a Clarcel prelayer, as described in example 2.

The polysaccharide K5 contained in the filtrate was precipitated from methanol, as described in example 2, and was then subjected to two treatments with hydrogen peroxide, as in example 4, followed by a third decolorization with hydrogen peroxide.

Finally, 39.5 g of polysaccharide K5 were obtained, with a titer of 81% and a yield of 71%.

### Example 6

Treatment of the Pellet and Improvement of the Purification

Only the supernatants were extracted and purified in the preceding tests. In order to recover the polysaccharide K5 present in the centrifugation sediments, the pellet from the fermenter COL5K002 of example 1 (5 kg containing 31.9 g of polysaccharide K5) was resuspended in a volume of demineralized water and the pH was adjusted to 7.2 with 5N NaOH. The mixture was then stirred for 2 hours at room temperature, and then centrifuged for 90 minutes at 4700×g.

The supernatant was treated with benzethonium chloride and the pellets were stirred in an acetate solution and then filtered through a sinter funnel equipped with a Clarcel prelayer, as described in example 2.

The polysaccharide K5 contained in the filtrate was precipitated from methanol, as described in example 2, and was then treated three times with hydrogen peroxide, as in example 5.

29.6 g of polysaccharide K5 were obtained, with a titer of 73% and a final yield of 68%.

### Example 7

Treatment of the Pellet and Improvement of the Purification

The pellet from the fermenter COL5K003 of example 1 (4.75 kg containing 28.5 g of polysaccharide K5) was resuspended in 4.75 liters of demineralized water and the pH was adjusted to 7.2 with 5N NaOH. The mixture was then stirred for 2 hours at room temperature, and then centrifuged for 2 hours at 4700×g.

The supernatant was treated with benzethonium chloride and the pellets were stirred in an acetate solution and then filtered through a sinter funnel equipped with a Clarcel prelayer, as described in example 2.

The polysaccharide K5 contained in the filtrate was precipitated from methanol, as described in example 2, and was then subjected to two treatments with hydrogen peroxide, as in example 4. 25.9 g of polysaccharide K5 with a titer of 69% and a yield of 63% were obtained.

### Example 8

Purification Comprising a Step of Enzymatic Deproteination

A step of enzymatic deproteination was performed in order to improve the purity of the final product.

45.5 g of polysaccharide K5, comprising 15.9 g derived from example 7 and 29.6 g derived from example 6, were resuspended in 1.8 liters of demineralized water.

After adjusting the pH to 7.0, 910 μl of liquid alcalase were added and the mixture was incubated for 5 hours at 60° C. with stirring.

23 g of salt and 7.2 liters of methanol were then added and the mixture was stirred for 30 minutes, and then centrifuged for 15 minutes at 4000×g.

The pellet was resuspended in 1.8 liters of demineralized water and treated with hydrogen peroxide, as described in example 3.

36.4 g of polysaccharide K5 with a titer of 89% and a yield of 98% were obtained.

### Example 9

Comparison of the Efficacy of Enzymatic Deproteination Compared with Decolorization

5 g of polysaccharide K5 derived from example 7 were treated either with hydrogen peroxide or with alkaline alcalase, in order to estimate the relative efficacy of the two treatments.

5 g of polysaccharide K5 derived from example 7 were thus resuspended in 200 ml of demineralized water and the solution was treated with hydrogen peroxide, as in example 3.

3.75 g of polysaccharide K5 with a titer of 76.9% and a yield of 83% were obtained.

In the same manner, 5 g of polysaccharide K5 derived from example 7 were resuspended in 200 ml of demineralized water. However, instead of being treated with hydrogen peroxide, this solution was heated to 60° C. and its pH was adjusted to 7. 100 μl of alcalase were then added thereto and the mixture was incubated for 5 hours at 60° C.

This mixture was then precipitated from methanol, as detailed in example 2.

3.62 g of polysaccharide K5 with a titer of 83% and a yield of 87% were obtained, compared with the titer of 76.9% and the yield of 83% obtained with the hydrogen peroxide treatment.

The deproteination step thus makes it possible to obtain a better yield and also greater purity compared with a third treatment with hydrogen peroxide.

At the end of the fermentation as described in example 1, the losses in the pellet were 60% in COL5K002 and 40% in COL5K003. The process for retreating this pellet developed and described in examples 6 and 7 made it possible to increase the overall yield for the extraction of polysaccharide K5 from 37% to 78% for COL5K002 and from 43% to 68% for COL5K003.

The final deproteination step described in example 8 makes it possible to bring the polysaccharide K5 purity to 89% with a quantitative yield (98%).

1. A process for preparing polysaccharide K5, comprising:
a) a step of fermentation, which may be performed in a fermenter, of a strain of Escherichia coli that produces polysaccharide K5, comprising:
a batch growth phase,
one or more fed batch growth phases,
a cooling phase, during which the temperature is decreased and the pH of the medium rises to a value that may be up to 9,
a phase of separating the biomass from the culture medium,
a phase of decontaminating the culture medium from the biomass,
b) a purification step using the culture medium and/or the biomass.

2. The process for preparing polysaccharide K5 according to claim 1, wherein the purification step starting with the culture medium and/or the biomass comprises:
adjustment of the pH to between about 7 and about 11,
a first precipitation,
dissolution of the precipitate,
filtration of the resulting suspension, and
precipitation from an alcohol.

3. The process according to claim 1, wherein it comprises two batch growth phases with exponential feed of substrate following the batch growth phase.

4. The process according to claim 1, wherein the cooling phase is preceded by a batch growth phase with constant feed of substrate.

5. The process according to claim 1, wherein the first fed batch growth phase begins on depletion of the carbon source in the medium during the batch growth phase.

6. The process according to claim 1, wherein:
the first fed batch growth phase begins on depletion of the carbon source in the medium during the batch growth phase,
where appropriate, the following fed batch growth phases begin when the concentration of dissolved oxygen drops significantly as a result of a limitation of the oxygen transfer.

7. The process according to claim 1, wherein:
the first fed batch growth phase begins on depletion of the carbon source in the medium during the batch growth phase,
where appropriate, the following fed batch growth phases begin when the concentration of dissolved oxygen drops significantly as a result of a limitation of the oxygen transfer,
the cooling phase begins when all the culture feed medium has been injected into the fermenter, or when a change in pH is detected.

8. The process according to claim 1, wherein the carbon source is either glucose or glycerol.

9. The process according to claim 1, wherein the carbon source is glycerol.

10. The process according to claim 1, wherein the carbon source is glycerol and its concentration is between about 10 and about 90 g/l in the initial culture medium for the batch growth phase, and between about 250 and about 1200 g/l in the feed medium for the fed batch growth phases.

11. The process according to claim 1, wherein the nitrogen source in the medium during the batch growth phase consists of yeast extracts, casamino acids, peptones, NH₄OH, NH₃ or (NH₄)₂HPO₄.

12. The process according to claim 1, wherein the nitrogen source in the initial culture medium during the batch growth phase is NH₄OH, NH₃ or (NH₄)₂HPO₄.

13. The process according to claim 12, wherein the concentration of the nitrogen source in the initial culture medium during the batch growth phase is between about 1 and about 10 g/l.

14. The process according to claim 1, wherein the nitrogen source is provided by NH₄OH or NH₃ for regulation of the pH during the batch growth and fed batch growth phases.

15. The process according to claim 1, wherein the nitrogen source is provided by NH₄OH or NH₃ at a concentration of about 20% for regulation of the pH during the batch growth and fed batch growth phases.

16. The process according to claim 1, wherein the nitrogen source in the medium during the batch growth phase is NH₄OH, NH₃ or (NH₄)₂HPO₄, at a concentration of between about 1 and about 10 g/l in the initial culture medium, and is provided by NH₄OH or NH₃ at a concentration of about 20% for regulation of the pH during the batch growth and fed batch growth phases.

17. The process according to claim 1, wherein the growth temperature is between about 10° C. and about 40° C.

18. The process according to claim 1, wherein the growth temperature is about 30° C. during the batch growth phase and the first fed batch growth phase, and, where appropriate, it is about 25° C. during the other fed batch growth phases.

19. The process according to claim 1, wherein:
the carbon source is glycerol at a concentration of between:
about 10 and about 90 g/l in the initial culture medium for the batch growth phase, and
about 250 and about 1200 g/l in the feed medium for the fed batch growth phases, and
the growth temperature is:
about 30° C. during the batch growth phase and the first fed batch growth phase, and
about 25° C., where appropriate, during the other fed batch growth phases.

20. The process according to claim 1, wherein:
the carbon source is glycerol at a concentration of between:
about 10 and about 90 g/l in the initial culture medium for the batch growth phase, and
about 250 and about 1200 g/l in the feed medium for the fed batch growth phases; and
the nitrogen source in the medium during the batch growth phase:
is NH₄OH, NH₃ or (NH₄)₂HPO₄, at a concentration of between about 1 and about 10 g/l in the initial culture medium, and
is provided by NH₄OH or NH₃ at a concentration of about 20% for regulation of the pH during the batch growth and fed batch growth phases.

21. The process according to claim 1, wherein:
the nitrogen source in the medium during the batch growth phase:
is NH₄OH, NH₃ or (NH₄)₂HPO₄, at a concentration of between about 1 and about 10 g/l in the initial culture medium, and
is provided by NH₄OH or NH₃ at a concentration of about 20% for regulation of the pH during the batch growth and fed batch growth phases; and
the growth temperature is:
about 30° C. during the batch growth phase and the first fed batch growth phase, and
about 25° C., where appropriate, during the other fed batch growth phases.

22. The process according to claim 1, wherein:
the fermentation step comprises the following steps, in the following order:
a batch growth phase,
a batch growth phase with exponential feed of substrate starting from the total depletion of the carbon source during the preceding phase,
a second batch growth phase with exponential feed of substrate starting when the oxygen concentration drops significantly as a result of a limitation of the oxygen transfer in the culture medium during the preceding phase,
a batch growth cycle with constant feed of substrate starting when the oxygen concentration drops significantly as a result of a limitation of the oxygen transfer in the culture medium during the preceding phase, and
a cooling phase during which the temperature is decreased to about 12° C. and the pH rises to a value of between about 7.5 and about 9,
a phase of decontamination of the culture medium from the biomass,
and is performed with the following parameters:
the temperature is:
about 30° C. during the batch growth and the first fed batch growth phase, and
about 25° C. during the other two fed batch growth phases;
the nitrogen source is provided by:
(NH₄)₂HPO₄, NH₄OH or NH₃ at a concentration of between about 1 and about 10 g/l in the initial culture medium,
the addition of NH₄OH or concentrated NH₃ to about 20% for regulation of the pH during the batch growth and fed batch growth phases,
the carbon source is glycerol:
at a concentration of between about 10 and about 90 g/l in the initial culture medium during the first batch growth phase, and
at a concentration of between about 250 and about 1200 g/l in the feed medium during the fed batch growth phases.

23. The process according to claim 1, wherein, after the batch growth phase, the fed batch growth phases and the cooling phase, the biomass is separated from the culture medium by centrifugation.

24. The process according to claim 1, wherein, after separation, the biomass and the culture medium are inactivated for a period ranging between about 1 hour and about 3 hours at a temperature of about 80° C.

25. The process according to claim 2, wherein the first precipitation of the purification step is performed using a quaternary ammonium salt.

26. The process according to claim 25, wherein the quaternary ammonium salt is benzethonium chloride.

27. The process according to claim 2, wherein the first precipitate from the purification step is redissolved in sodium acetate, at a concentration of 10%.

28. The process according to claim 2, wherein the filtrate from the purification step is precipitated from methanol.

29. The process according to claim 2, wherein the first precipitation of the purification step is performed using benzethonium chloride, this precipitate is redissolved in sodium acetate, it is filtered off, and the filtrate from the purification step is precipitated from methanol.

30. The process according to claim 1, wherein the product after purification is decolorized one or more times with hydrogen peroxide.

31. The process according to claim 30, wherein the product after purification is decolorized three times with hydrogen peroxide.

32. The process according to claim 1, wherein the product after purification and three decolorizations with hydrogen peroxide undergoes another decolorization with hydrogen peroxide.

33. The process according to claim 1, further comprising a step of treatment with a protease.

34. The process according to claim 33, wherein the protease used is alcalase.

35. The process according to claim 1, wherein the purification step comprises the following steps:
centrifugation of the culture to separate the culture medium from the biomass;
using the supernatant from the centrifugation of the fermentation broth for:
adjustment of the pH to between about 7 and about 11,
precipitating with benzethonium chloride,
washing the precipitate,
dissolving the precipitate in 10% sodium acetate,
filtering the 10% sodium acetate solution,
precipitating the filtrate from methanol,
decolorizing the filtrate three times with hydrogen peroxide,
treating with alcalase;
in parallel, using the pellet resulting from the centrifugation of the fermentation broth for:
resuspending the pellet,
adjusting the pH to between about 7 and about 11,
precipitating with benzethonium chloride,
washing the precipitate,
redissolving the precipitate in 10% sodium acetate,
filtering the 10% sodium acetate solution,
precipitating the filtrate from methanol,
decolorizing the filtrate three times with hydrogen peroxide,
treating with alcalase;
combining the two preparations in any one of the purification steps;
precipitating, centrifuging and drying the polysaccharide K5.

36. The process according to claim 1, wherein:
the fermentation step:
comprises the following steps, in the following order:
a batch growth phase,
a batch growth phase with exponential feed of substrate starting from the total depletion of the carbon source during the preceding phase,
a second batch growth phase with exponential feed of substrate starting when the oxygen concentration drops significantly as a result of a limitation of the oxygen transfer in the culture medium during the preceding phase,
a batch growth cycle with constant feed of substrate starting when the oxygen concentration drops significantly as a result of a limitation of the oxygen transfer in the culture medium during the preceding phase, and
a cooling phase during which the temperature is decreased to about 12° C. and the pH rises to a value of between about 7.5 and about 9,
a phase of decontamination of the culture medium from the biomass,
and is performed with the following parameters:
the temperature is:
about 30° C. during the batch growth and the first fed batch growth phase, and
about 25° C. during the other two fed batch growth phases;
the nitrogen source is provided by:
(NH₄)₂HPO₄, NH₄OH or NH₃ at a concentration of between about 1 and about 10 g/l in the initial culture medium,
the addition of NH₄OH or concentrated NH₃ to about 20% for regulation of the pH during the batch growth and fed batch growth phases,
the carbon source is glycerol:
at a concentration of between about 10 and about 90 g/l in the initial culture medium during the first batch growth phase, and
at a concentration of between about 250 and about 1200 g/l in the feed medium during the fed batch growth phases;
and the purification step comprises the following steps:
centrifugation of the culture to separate the culture medium from the biomass;
using the supernatant from the centrifugation of the fermentation broth for:
adjustment of the pH to between about 7 and about 11,
precipitating with benzethonium chloride,
washing the precipitate,
dissolving the precipitate in 10% sodium acetate,
filtering the 10% sodium acetate solution,
precipitating the filtrate from methanol,
decolorizing the filtrate three times with hydrogen peroxide,
treating with alcalase;
in parallel, using the pellet resulting from the centrifugation of the fermentation broth for:
resuspending the pellet,
adjusting the pH to between about 7 and about 11,
precipitating with benzethonium chloride,
washing the precipitate,
redissolving the precipitate in 10% sodium acetate,
filtering the 10% sodium acetate solution,
precipitating the filtrate from methanol,
decolorizing the filtrate three times with hydrogen peroxide,
treating with alcalase;
combining the two preparations in any one of the purification steps;
precipitating, centrifuging and drying the polysaccharide K5.

37. A process, comprising: treating a polysaccharide prepared according to claim 1, with at least one treatment selected from: enzymatic deacetylation, chemical deacetylation, N-resulfatation, C-5 epimerization, or O-sulfatation reaction.

38. The process according to claim 37, wherein a bioheparin results.

39. The process according to claim 38, further comprising: treating the bioheparin as substrate in a fragmentation reaction to obtain an LMWH (low molecular weight heparin), the molecular weight of which is between about 1500 and about 6500 Da.

40. The process according to claim 39, wherein the LMWH is a low molecular weight heparin with a molecular weight of between about 3500 and about 5500 Da, or a very low molecular weight heparin with a molecular weight more of between about 1500 and about 3000 Da.
